(19) United States
(12) Patent Application Publication
(10) Pub. No.: US 2024/0108226 A1
(43) Pub. Date: Apr. 4, 2024

(54) MEDICAL DEVICE DISTAL OPTICS POTTING

(71) Applicant: Canon U.S.A., Inc., Melville, NY (US)

(72) Inventor: Mark Alan Hamm, Lynnfield, MA (US)

(21) Appl. No.: 17/936,791

(22) Filed: Sep. 29, 2022

(51) Int. Cl. A61B 5/00

(52) U.S. Cl. CPC A61B 5/0084; A61B 5/6852

(57) ABSTRACT

A medical device includes a flexible member having a hollow cavity extending between a proximal end and a distal end, an optical fiber extending through the hollow cavity, and a potting material to secure/pot the optical fiber to the distal end to inhibit damage of the distal end. The potting material can be an adhesive, such as a UV curable adhesive or dual-cure adhesive. The medical device can be stripped or a buffered optical fiber, and can include a spacer, wherein the optical fiber can be fused to the spacer, and potted in the distal end. The medical device can have a rebuffer heat shrink tube surrounding the area of the optical fiber on a guidewire, and can have distal optics at the distal end that include at least a spacer, a lens and a reflector. The medical device can be a disposable catheter, and an MMOCT catheter.

### BACKGROUND

### Field of the Disclosure

The present disclosure generally relates to medical devices and, more particularly, to secure/pot components in catheter-based medical devices via potting.

Potting generally involves filling any voids in an assembly with material to give components support, seal from exposure, and strengthen an area of a device.

### Description of the Related Art

Medical devices including a catheter, endoscope, bronchoscope, laparoscope, ablation device, and other devices carry out medical procedures where a flexible medical tool or hollow tube is inserted into a patient's body and an instrument is passed through the tool to examine or treat an area inside the body.

A disposable medical device in the form of an MMOCT fiber-optic imaging catheter can work reliably a number of times in a single patient, six or more, for example.

Optical fiber-based catheters often employ distal optics at the distal, usable end, where the fiber buffer layer is often stripped off to allow fusing and other connecting operations. The MMOCT catheter sometimes breaks at a stripped fiber region near the distal end, resulting in catheter replacement. If the catheter breaks and stops working correctly, the medical procedure can be delayed which adds risk to the patient. The process of replacing a catheter that has been inserted into a sensitive, potentially disrupted artery adds significant and undue risk to the patient. This is particularly problematic in very tortuous anatomy, presumably due to optical fiber flexural stress at the stripped region near the distal optics.

It would be beneficial to extend the longevity of the device and avoid these failures.

### SUMMARY

The present disclosure advantageously inhibits damage, breakage, replacement, etc., and promotes and extends the lifetime and/or longevity of a medical device, optical fiber, catheter, other components, or combinations thereof.

According to some embodiments, a medical device includes a flexible member having a hollow cavity extending between a proximal end and a distal end, an optical fiber extending through the hollow cavity, and a potting material to secure/pot the optical fiber and distal optics to the distal end to inhibit damage of the distal end. The potting material can be an adhesive, wherein the adhesive can be UV curable adhesive, a dual-cure adhesive, or another adhesive.

According to some embodiments, the medical device can be stripped or a buffered optical fiber, and can include a spacer, wherein the optical fiber can be fused to the spacer, and potted in the distal end. The medical device can have a rebuffer heat shrink tube surrounding the area of the optical fiber with a gap between the optical fiber and heat shrink tube filled with potting material. The potting material can be a dual-cure adhesive inside the rebuffer heat shrink tube only. The optical fiber with the rebuffer heat shrink tube can be on a guidewire. The medical device can have distal optics at the distal end that include at least a spacer, a lens and a reflector. The medical device can be a disposable catheter, and an MMOCT catheter. The medical device can functionally interact with a display to display images, data or other information.

Further features of the present disclosure will become apparent from the following description of exemplary embodiments with reference to the attached drawings, where like structure is indicated with like reference numerals.

### BRIEF DESCRIPTION OF THE DRAWINGS

FIGS. 1 and 2 illustrate stripped optical fiber regions of a catheter according to some embodiments.

FIG. 3 illustrates a catheter according to some embodiments.

FIG. 4 illustrates a medical arrangement according to some embodiments.

### DESCRIPTION OF THE EMBODIMENTS

Various exemplary embodiments, features, and aspects of the disclosure will be described with reference to the drawings.

In the following embodiments, medical device and apparatus configurations are described that include a catheter, endoscope, bronchoscope, ablation device, other devices, and combinations thereof that may have different performance or operational characteristics, parameters, advantages, disadvantages, or the like. The present disclosure is not limited to any particular configuration.

Medical devices according to some embodiments carry out medical procedures where a flexible medical tool or hollow tube is inserted into a patient's body and an instrument is passed through the tool to examine or treat an area inside the body. The medical devices and procedures can provide a minimally invasive look into a hollow viscous or body cavity, as well as information through optical coherence tomography (OCT), multimodal, multimodality or multi-modality OCT (MMOCT), in vivo imaging, intravascular imaging, percutaneous coronary intervention (PCI), coronary angiography, intravascular ultrasound (IVUS), radiology, ultrasonography, other imaging modalities, and combinations thereof. The medical devices can have an adjustable tip that can be controlled remotely to alter the field of view without moving the rest of the device.

Multimodal optical imaging combines strengths of different imaging modalities across various physical contrast mechanisms, provides comprehensive structural, functional and molecular information of living tissue, and a medical device according to some embodiments can be used for cardiology, ophthalmology, coronary artery dilation, urethra insertion, laparoscopic surgery, vascular injection, angioplasty, angiography, atherosclerosis, venous and arterial thrombosis, vascular disease, vascular injury, other medical procedures, and combinations thereof.

The medical device is disposable and can be a single-use device to reduce cost and environmental waste, and certain types of devices can be safely reused as a multi-use, re-use, or reusable device when adequately cleaned, disinfected, sterilized, etc.

Some embodiments functionally interact with a continuum robot, robotic or snake catheter assembly with a rotational drive assembly or other actuator to impart rotational movement to an imaging core of the medical device or instrument including an optical catheter, steerable catheter, endoscope, or other flexible medical device or surgical tool.

A fiber optic catheter is an example of an optical catheter that includes a flexible sheath with a coil, optical probe and/or imaging core contained within the sheath, and can include other components. The catheterization procedure can include a ‘guide catheter’ which functions to help guide guidewires and other catheter-based devices into the ostium, the opening to the coronary arteries. The drive assembly is referred to as a patient interface unit (PIU), and can be releasably connected to the catheter and a breakaway mechanism can be used so the drive assembly disconnects from the catheter in response to a breakaway force.

A disposable MMOCT fiber optic imaging catheter can work reliably a number of times in a single patient, six or more, for example.

If the catheter breaks and stops working correctly, the medical procedure can be delayed which adds risk to the patient. The process of replacing a catheter that has been inserted into a sensitive, potentially disrupted artery adds significant risk to the patient.

Advantageous features, characteristics, and other attributes according to some embodiments inhibit damage, breakage, replacement, etc., and promote and extend the lifetime and/or longevity of the medical device, optical fiber, catheter, other components, or combinations thereof.

A medical device according to some embodiments include various configurations of a catheter with an elongate, flexible, tubular member extending between proximal and distal ends that is configured for intravascular placement within an internal lumen.

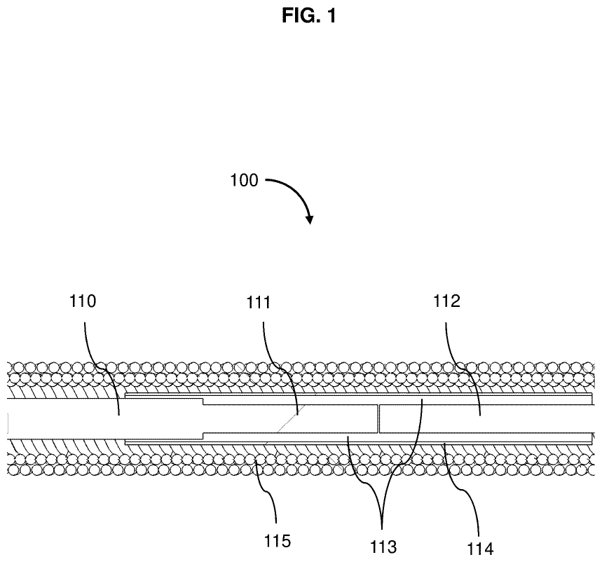

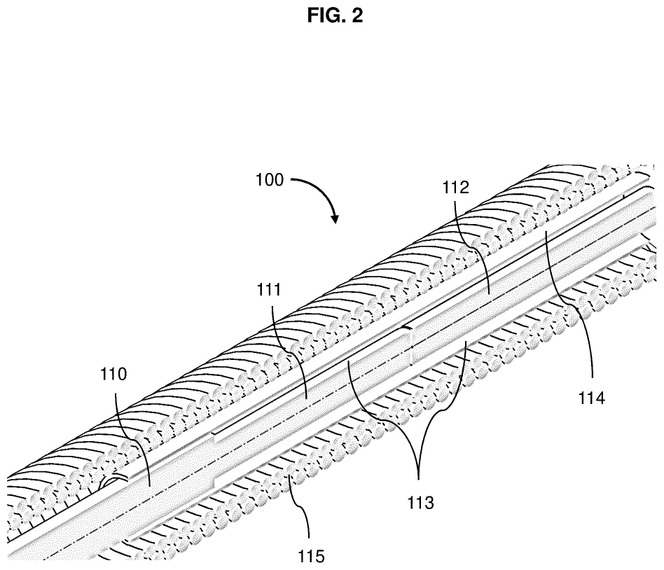

FIGS. 1 and 2 show a catheter 100 according to some embodiments including an optical fiber, an optical fiber buffer, a dual-cure adhesive, a rebuffer, and distal optics at the distal end. A stripped optical fiber region of the catheter 100 is illustrated and includes a buffered optical fiber region 110, a stripped optical fiber region 111, a spacer 112, adhesive 113, a rebuffer heat shrink tube 114, and a drive cable 115, and other components can be included.

The buffered optical fiber region no protects the optical fiber region 111 that is a portion of the optical fiber where the buffer layer has been stripped. The adhesive 113 can secure/pot the stripped optical fiber region 111 and the spacer 112 to inhibit damage to the optical fiber region 111. The rebuffer heat shrink tube 114 covers the stripped optical fiber region 111. The rebuffer heat shrink tube 114 can surround the stripped area of the optical fiber region 111 with a gap between the optical fiber region 111 and rebuffer heat shrink tube 114, and can be filled with potting material. The rebuffer tube can be used on or around the stripped region after fusing to wick dual-cure adhesive into the rebuffer tube to form a seal and surround the bare glass of the fiber in the stripped region. Preferably, the dual-cure adhesive is inside the rebuffer tube only to form or reform a tight buffer layer and preserve integrity of optical fiber glass near the fused or bonded joint.

The drive cable 115 is coiled around the optical fiber region 111 to drive the catheter 100 and rotate the optical fiber in during imaging procedures.

Potting is used according to some embodiments to fill any voids in the optical fiber region or other areas of the catheter with material to give components support, seal from exposure, and strengthen an area of the medical device.

Coating materials to coat and buffer the optical fiber region no are selected based on parameters including the modulus of elasticity or Young's Modulus, index of refraction, temperature range, viscosity and cure speed, adhesion and resistance to delamination, stripability, microbending performance, abrasion resistance, other parameters, or combinations thereof. The coating materials generally do not to come off while in use, but the ability to remove short lengths of coating when not in use is useful for splicing, mounting connectors, making fused connections, or the like where very high temperatures are used that melt or fuse the glass together.

Suitable coating materials include various glasses, acrylics, acrylates, polymers, polymer coating compositions, polyimides, carbon, metals, nitrides, sapphire, silicone, dyes, fluorescent materials, sensing reagents, nanomaterials, other materials, or combinations thereof. The coating materials protect the surface of the silica or glass core and cladding of the optical fiber from air, moisture, chemical contaminants, nicks, bumps, abrasions, tight bends, microcracks, and other hazards, which can cause flaws in the glass surface.

An ultraviolet (UV) cured acrylic polymer coating material protects the optical fiber from harmful radiation effects and increases the outside diameter of the optical fiber region 111. The UV-cured coating can be a two-layer coating with a primary coating and a secondary coating. The primary coating can be a softer inner layer and the secondary coating can be a harder outer layer. The coatings can have different optical and mechanical characteristics.

The optical fiber region 111 can have multiple layers including a core, at least one cladding layer, at least one coating layer, a buffer layer, an outer jacket, other layers, components, performance characteristic attributes, hybrids or combinations thereof. The core is a cylinder of quartz, silica, glass, plastic, or other material, and provides a light transmitting medium to guide the light. The cladding surrounds the core for trapping light in the core, and the optical fiber is protected by the buffer layer, coating layer, outer jacket, other layers, or combinations thereof which surround the cladding. Laser radiation or optical energy passes through the core between the proximal and distal ends to a target as a result of internal refraction between the core and cladding glass layers. Light energy that escapes the core is bent back into the core by the lower refraction index of the cladding layer. The buffer layer can be a polymer layer tightly surrounding the optical fiber glass. The buffer strengthens and toughens the optical fiber.

The buffer layer is removed from the distal end of the optical fiber forming the stripped optical fiber in, which is fused to a coreless fiber to act as the spacer 112. The coreless fiber lacks a core so the light beam dissipates instead of being guided through the core.

The stripped optical fiber region 111 is a portion of the optical fiber where the buffer layer has been stripped. The outer diameter of the stripped optical fiber region 111 is smaller or reduced from the outer diameter of the buffer region no. The removal of the outer buffer layer creates a weak, unsupported area of fiber because the jacket supports the glass fiber in bending and flexure, but additionally exposes the glass fiber to ambient humidity which can cause micro-cracks on the fiber outer surface at the exposed area.

The stripped optical fiber region 111 is at the distal end and can include the spacer 112. The adhesive 113 acts as a potting that is used to secure or pot the stripped optical fiber region and the fused spacer 112. If high sensitivity is not needed, the spacer 112 can be bonded to the optical fiber in using adhesive, but this can create unwanted back reflections due to refractive index mismatch. Additional optical components can include a lens and reflector that can be potted within the catheter 100, where all potted components should have a greater fatigue life than without potting. Since the optical fiber contributes most of the tensile strength of the core, potting inhibits damage of the distal end and also serves as a strong attachment to the distal end for ultimate tensile strength.

Potting is used according to some embodiments to fill any voids in the optical fiber region or other areas of the catheter with material to give components support, seal from exposure, inhibit damage, and strengthen an area of the medical device.

The stripped area of the optical fiber region 111 can be strengthened through annealing the fiber after the optical fiber region 111 is fused to the spacer 112. The optical fiber region 111 and spacer 112 should locally melt in order to be fused together. The annealing of the fiber leads to a lower stressed region that becomes stronger and has a higher flexural strength and fatigue resistance.

Annealing the optical fiber in includes heating to near melting temperature and then cooling the fiber very slowly, processes that can be controlled by a fiber fusing machine.

The heat shrink tube 114 is a rebuffer and is placed over the stripped optical fiber region 111. The adhesive 113 can be wicked into the heat shrink tube 114 and over and around the stripped portion of the optical fiber in. The rebuffer heat shrink tube 114 can surround the stripped area of the optical fiber region 111 with a gap between the optical fiber region 111 and rebuffer heat shrink tube 114 filled with potting material.

The heat shrink tube 114 is formed of suitable material including FEP Teflon™, PET balloon tubing, flexible Pebax®, or other materials. The heat shrink tube is not heated or shrunk onto the substrate, but is left expanded so that there is space between it and the fiber, spacer, etc. for adhesive to wick in and surround and seal the glass components. The adhesive 113 can be a UV adhesive and effectively reforms the outer buffer layer that supports the optical fiber region 111 once it is fully cured, it thereby effectively increases the tolerance to flexural fatigue and the working life of this area of the optical fiber. Preferably, the dual-cure adhesive is inside the rebuffer tube only to form or reform a tight buffer layer and preserve integrity of optical fiber glass near the fused or bonded joint.

The heat shrink tube 114 is used as a re-coating material that simulates the original buffer layer or fiber buffer coating that is removed prior to fusing the optical fiber region 111 to the spacer 112 due to the high heat needed to melt glass.

To prevent micro-cracking glass in the optical fiber region 111, ambient humidity should be kept relatively low to avoid weakening the optical fiber as it is exposed to air, and particularly humid air. Humidity is known to negatively affect fiber longevity due to its contribution to the formation of micro-cracks at the surface of the optical fiber. Micro-cracks can propagate with repeated flexure, so early fiber fatigue failure can be an issue, particularly in imaging catheters where the core and optical fiber rotate at high speeds. Since the catheter core can rotate at very high speeds, optical fiber fatigue failure is a factor especially in tortuous anatomy where the catheter is bent. When an optical fiber is rotated at high speeds while the stripped area of the optical fiber is in a bend, that optical fiber is effectively flexed back and forth during each revolution. This can quickly make fiber fatigue an issue, and micro-cracks can quickly propagate, forming larger, stress-focusing cracks that lead to premature fiber failure.

Since a PCI procedure can be delayed should the catheter fail, it is acceptable to spend considerable resources to increase fatigue life and prevent premature catheter failure that could harm the patient.

The drive cable 115 is coiled around the optical fiber in and is connected to the PIU via a proximal connector to drive the catheter 100 and rotate the optical fiber during imaging procedures.

The catheter 100 can include a plurality of optical fibers or fiber optics where each optical fiber 111 can be a single mode or multimode optical fiber and the catheter 100 can be a disposable or multi-use MMOCT fiber-optic imaging catheter. The catheter 100 can work reliably as a disposable or multi-use catheter a number of times in a patient, six or more, for example. Certain types of disposable or multi-use medical devices can be reused many times after sanitization using a chemical agent composition such as Cydex or other compositions.

The optical fiber region 111 can be configured as an optical probe to provide the catheter 100 with the ability to sense vessel and blood characteristics. The catheter 100 can rotate the optical probe for circumferential scanning and have distal optics or imaging components in the distal end that include a spacer, a lens for focusing, and a reflector. The catheter 100 can also have a prism, mirror, an optical light source, a light emitting diode (LED), an optical detector, a gradient index (GRIN) lens, a guidewire, a guidewire entry, other components, or combinations thereof. The guidewire can be inserted through the catheter 100 to guide the catheter 100. The lens for focusing can be attached to the spacer to focus the light energy into a defacto beam, whose diameter is related to lateral resolution.

The lens can be a GRIN lens, a fiber Bragg lens, a ball lens, other types of lenses, or combinations thereof. A sheath carries the optical fiber in and the prism or mirror can direct light. The proximal end has a free space that allows the catheter 100 to rotate while keeping the proximal end fixed. One or more motors can rotate the catheter 100 and can be formed as a rotary junction, such as a fiber optic rotary joint (FORJ) or the like. The lens, mirror and other optical focusing and aiming components can be mounted in the distal tip or other areas of the catheter 100.

The catheter 100, optical fiber core, cladding, buffer layer, coating layer, outer jacket, and other characteristic attributes are subject to becoming damaged or weakened over time due to deterioration, degradation, flexural stress, other reasons, or combinations thereof, which can result in or lead to premature breakage, replacement, or the like.

In a disposable or multi-use MMOCT fiber-optic imaging catheter, for example, optical fibers can sometimes break at a stripped region near the distal end, resulting in catheter replacement. If the catheter breaks and stops working correctly, the medical procedure can be delayed which adds risk to the patient. The process of replacing a catheter that has been inserted into a sensitive, potentially disrupted artery adds significant and undue risk to the patient. This is particularly problematic in very tortuous anatomy, presumably due to optical fiber flexural stress at the stripped region near the distal optics, or at adhesive-bonded joints in the optical path.

When a bare glass optical fiber is exposed to ambient conditions including relatively high humidity, such as in the stripped region to be fused, micro-cracks can form at or near the surface of the fiber. During use, tortuous anatomy can flex the fiber, which can cause the micro-cracks to propagate and grow in size, leading eventually to fiber breakage. The prevention of micro-cracks is important for catheter longevity because the distal tip of the medical device operates in tortuous anatomy while rotating at speeds or frequencies around 200 Hz. This effectively flexes the distal end of the device, including the optical fiber, 200 times per second during imaging, which accelerates the rate of micro-crack growth, which can lead to significant stress risers that lead to premature optical fiber breakage, which renders the device inoperable.

The rebuffer tube can be used on or around the stripped region after fusing to wick dual-cure adhesive into the rebuffer tube to form a seal and surround the bare glass of the fiber in the stripped region. The rebuffer heat shrink tube 114 can surround the stripped area of the optical fiber region 111 with a gap between the optical fiber region 111 and rebuffer heat shrink tube 114 filled with potting material. Preferably, the dual-cure adhesive is inside the rebuffer tube only to form or reform a tight buffer layer and preserve integrity of optical fiber glass near the fused or bonded joint.

This provides benefits that include (1) potting the stripped region of the fiber, thusly protecting the bare fiber glass from exposure to the elements; (2) re-forming a buffer layer on the stripped region to help support the bare fiber glass in flexure, spreading stress over a longer length to help avoid concentrated flexural stress which can lead to premature fiber fatigue failure; (3) preventing the adhesive from contacting the drive cable coils, which would change and degrade drive cable performance; and can include other benefits.

Fatigue strength and resistance to breakage of the catheter 100 can be increased through effective control of the manufacturing area ambient conditions to keep the humidity low, to minimize the elapsed time that the stripped fiber is exposed to ambient conditions, and to re-coat or rebuffer the stripped fiber with the adhesive 113 in the form of the rebuffer material to act as a strain relief to minimize stress on the stripped fiber.

The rebuffer material of the adhesive 113 can be a UV curable adhesive or dual-cure adhesive, and the material is referred to as rebuffer material because the outer layer of acrylic or polyimide coating is called the buffer layer. Potting is used according to some embodiments to fill any voids in the optical fiber region or other areas of the catheter with material to give components support, seal from exposure, inhibit damage, and strengthen an area of the medical device. The use of a dual-cure potting material is suggested according to some embodiments to ensure that all of the adhesive fully cures, even in areas not exposed to UV curing light. Preferably, the dual-cure adhesive can be inside the rebuffer tube only to form or reform a tight buffer layer and preserve integrity of optical fiber glass near the fused or bonded joint.

The UV adhesive or dual-cure adhesive is formulated from epoxy, acrylate, other formulations, or combinations thereof to form strong and reliable bonds when exposed to light source after application. A dual-cure UV and secondary heat-curable-adhesive combines fast UV curability of acrylate composition and high adhesion performance of thermal cure epoxy composition, and can block UV, visible, near infrared, or other types of light. The light blocking property of the dual-cure UV or heat adhesive do not fade with time or heat, and facilitate precision assembly of components through on-demand curing and setting. The dual-cure adhesive is used with materials that transmit visible light, UV light, or other types of light, where cure times can be adjusted based on the type of application. The UV adhesive or dual-cure adhesive provides high strength, excellent stability, high cure speeds, on-demand curing, high viscosity, high transparency, high precision, secondary heat cure for areas not exposed to UV curing light, and other characteristic features.

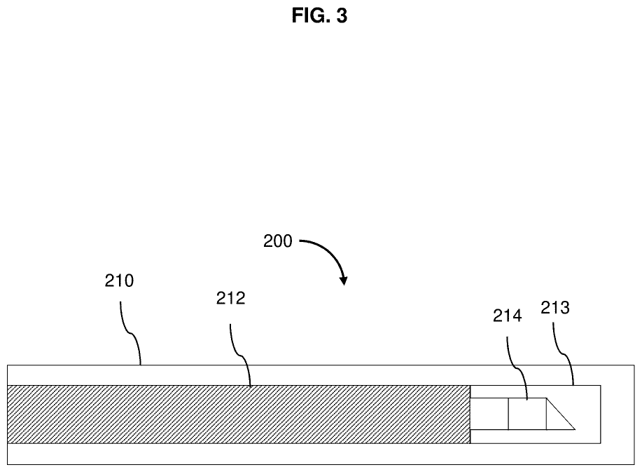

FIG. 3 shows a catheter 200 according to some embodiments that is similar to the catheter 100. The catheter 200 has a sheath 210, a coil 212, a protector 213, and an optical fiber 214. The catheter 200 has the components of the catheter 100 of a buffered optical fiber region, a stripped optical fiber region, a spacer, adhesive, a rebuffer heat shrink tube, a drive cable, and other components can be included. The catheter 200 can include an optical fiber buffer, a dual-cure adhesive, a rebuffer, and distal optics at the distal end. The optical fiber 214 can be configured as an optical probe to provide the catheter 200 with the ability to sense vessel and blood characteristics. The catheter 200 can rotate for circumferential scanning and can have distal optics at the distal end that include a spacer, a lens for focusing, and a reflector. The catheter 100 can also have a prism, mirror, an optical light source, an LED, an optical detector, a GRIN lens, a guidewire, a guidewire entry, other components, or combinations thereof. The guidewire can be inserted through the catheter 100 to guide the catheter 100. The lens for focusing can be attached to the spacer to focus the light energy into a defacto beam, whose diameter is related to lateral resolution.

The lens can be a GRIN lens, a fiber Bragg lens, a ball lens, other types of lenses, or combinations thereof. A sheath carries the optical fiber in and the prism or mirror can direct light. The proximal end has a free space that allows the catheter 100 to rotate while keeping the proximal end fixed. One or more motors can rotate the catheter 100 and can be formed as a rotary junction, such as an FORJ or the like. The lens, mirror and other optical focusing and aiming components can be mounted in the distal tip or other areas of the catheter 200.

Advantageous features, characteristics, and other attributes according to some embodiments inhibit damage, breakage, replacement, etc., and promote and extend the lifetime and/or longevity of the optical fiber, catheter, medical device, and/or other components.

The optical fiber based medical devices including the MMOCT catheter according to some embodiments become effective highly accurate measurement instruments that medical practitioners including cardiologists can rely upon for critical information that guides medical procedures including PCI procedures and other procedures. Medical devices according to some embodiments can be precisely assembled in a way that ensures that the end product remains precise and repeatable from catheter to catheter, so the information that they provide is reliable. Moreover, each catheter can be calibrated on the system to be used to run it prior to operation.

Assembly of the distal optics also ensures these desirable attributes. The utilization of UV curable adhesives that also cure via a secondary exposure to heat provides the ability to lock parts in place quickly and efficiently, ensuring that even areas of adhesive that are not exposed to UV curing energy will cure when exposed to sufficient heat.

The utilization of the dual cure adhesive and the UV plus available heat-based cure schedule is effective for the shadowed areas of adhesive cured in the catheter 100 when exposed to heat following initial, desirable tacking of components via UV, cure as assembly.

The stripped area of optical fiber near the MMOCT distal optics is such an area that is shielded from exposure to UV cure light. Adhesives with a higher durometer or Shore hardness would be suitable according to some embodiments, where durometer or Shore durometer is a standardized way to measure the hardness of materials. High durometer adhesives with Shore D 80 or higher durometer are suitable according to some embodiments because the small diameter within the unshrunk heat shrink rebuffer tube covering the stripped fiber region provided that they do in fact fully cure so that they can provide potting, support and reinforcement of the bare fiber.

A dual-cure UV and secondary heat-curable-adhesive combines fast UV curability of acrylate composition and high adhesion performance of thermal cure epoxy composition, and can block UV, visible, near infrared, or other types of light. The light blocking property of the dual-cure UV or heat adhesive do not fade with time or heat, and facilitate precision assembly of components through on-demand curing and setting. The dual-cure adhesive can be used with materials that transmit visible light, UV light, or other types of light, where cure times can be adjusted based on the type of application. The UV adhesive or dual-cure adhesive provides high strength, excellent stability, high cure speeds, on-demand curing, high viscosity, high transparency, high precision, secondary heat cure for areas not exposed to UV curing light, and other characteristic features.

Potting is used according to some embodiments to fill any voids in the optical fiber region or other areas of the catheter with material to give components support, seal from exposure, and strengthen an area of the medical device. The use of a dual-cure adhesive as a potting material is suggested according to some embodiments to ensure that all of the adhesive fully cures, even in areas not exposed to UV curing light. Preferably, the dual-cure adhesive is inside the rebuffer tube only to form or reform a tight buffer layer and preserve integrity of optical fiber glass near the fused or bonded joint.

The UV adhesive bonds well to both the acrylic optical fiber jacket, and also the proximal end of the spacer, a glass rod creating a functional ‘strain relief’ for the stripped distal end of the optical fiber.

Also, since the optical fiber acts as the main tensile member in the medical device, any excess frictional resistance to core movement could significantly increase stress on the fiber. As the PIU pulls back the core, tensile forces are transferred to the distal tip of the core via the optical fiber.

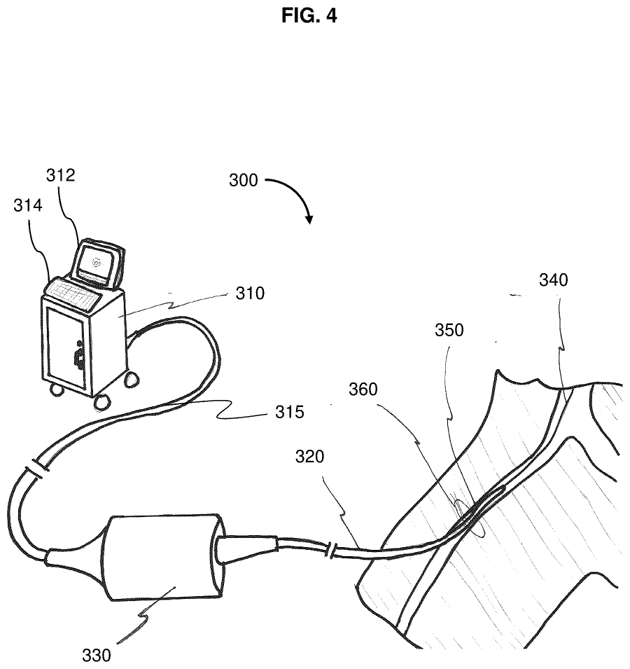

FIG. 4 shows a medical arrangement 300 that can functionally interact with the medical device or catheter configurations according to some embodiments.

The medical arrangement 300 has an imaging console 310, an imaging catheter 320, a motor drive 330, and an imaging catheter 330, a guidewire 340, and can include other components. The imaging catheter 330 is removably connected to the imaging console 310 through the motor drive 320 and interconnecting cabling and connectors 315. The imaging catheter 330 is on the guidewire 340 and includes distal optics that pass through the blood vessel or lumen 350 with the guidewire 340. The guidewire 340 is inserted through the catheter 330 to guide the catheter 330. The catheter 330 is driven by the motor drive 320 that is connected to the imaging console 310. The imaging catheter 330 obtains images and imagery while rotating about and extending an imaging plane 360 back and forth in the blood vessel 350 during pullback.

The imaging console 310 includes one or more or a combination of a processor, controller, control circuitry, memory, an input and output (I/O) interface, a communication interface, and can include other elements or components, and is configured to perform overall control of the medical arrangement 300. The imaging console 310 can include a display 312 and keyboard 314 to facilitate user interaction with the console through a graphical user interface (GUI), and be interconnected with medical instruments or other devices, and can be controlled independently, externally, or remotely by a controller. The display 312 can present a display to a user to view images, data or other information, and can be configured as a liquid crystal display (LCD), LED or other type of display.

As described above, advantageous features, characteristics, and other attributes according to some embodiments inhibit damage, breakage, replacement, etc., and promote and extend the lifetime and/or longevity of the medical device, optical fiber, catheter, other components, or combinations thereof.

The medical device according to some embodiments includes a flexible member having a hollow cavity extending between a proximal end and a distal end, an optical fiber extending through the hollow cavity, and a potting material to secure/pot the optical fiber to the distal end to inhibit damage to the distal end.

The potting material can be an adhesive including a UV curable adhesive or a dual-cure adhesive via secondary heat cure.

The medical device can be stripped or a buffered optical fiber, and can include a spacer, wherein the optical fiber can be fused to the spacer, and potted in the distal end. The medical device can have a rebuffer heat shrink tube surrounding the area of the optical fiber with a gap between the optical fiber and heat shrink tube filled with potting material. The potting material can be a dual-cure adhesive inside the rebuffer heat shrink tube only. The optical fiber with the rebuffer heat shrink tube can be on a guidewire. The medical device can have distal optics at the distal end that include at least a spacer, a lens and a reflector. The medical device can be a disposable catheter, and an MMOCT catheter. The medical device can functionally interact with a display to display images, data or other information.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

What is claimed is:

1. A medical device comprising:
a flexible member having a hollow cavity extending between a proximal end and a distal end;
an optical fiber extending through the hollow cavity; and
a potting material to secure/pot the optical fiber and distal optics to the distal end to inhibit damage of the distal end.

2. The medical device according to claim 1, wherein the potting material is an adhesive.

3. The medical device according to claim 2, wherein the adhesive is an ultraviolet (UV) curable adhesive.

4. The medical device according to claim 3, wherein the UV curable adhesive is a dual-cure adhesive via secondary heat cure.

5. The medical device according to claim 1, wherein the optical fiber is stripped or is a buffered optical fiber.

6. The medical device according to claim 1, further comprising a spacer,
wherein the optical fiber is fused to the spacer, and is potted in the distal end.

7. The medical device according to claim 1, further comprising a rebuffer heat shrink tube surrounding the stripped area of the optical fiber with a gap between the optical fiber and heat shrink tube filled with potting material.

8. The medical device according to claim 7, wherein the potting material is a dual-cure adhesive inside the rebuffer heat shrink tube only.

9. The medical device according to claim 7, wherein the optical fiber with the rebuffer heat shrink tube is on a guidewire.

10. The medical device according to claim 1, wherein the distal optics comprise at least a spacer, a lens and a reflector.

11. The medical device according to claim 1, wherein the medical device is a disposable catheter.

12. The medical device according to claim 11, wherein the catheter is a multimodal optical coherence tomography catheter.

13. The medical device according to claim 1, wherein the medical device functionally interacts with a display to display images, data or other information.

14. A medical apparatus comprising:
a processor; and
a medical device, the medical device including
a flexible member having a hollow cavity extending between a proximal end and a distal end;
an optical fiber extending through the hollow cavity; and
a potting material to secure/pot the optical fiber and distal optics to the distal end to inhibit damage of the distal end.

15. The medical apparatus according to claim 14, wherein the potting material is an adhesive.

16. The medical apparatus according to claim 14, wherein the adhesive is an ultraviolet (UV) curable adhesive.

17. The medical apparatus according to claim 16, wherein the UV curable adhesive is a dual-cure adhesive via secondary heat cure.

18. The medical apparatus according to claim 14, wherein the optical fiber is stripped or is a buffered optical fiber.

19. The medical apparatus according to claim 14, further comprising a spacer,
wherein the optical fiber is fused to the spacer, and is potted in the distal end.

20. The medical apparatus according to claim 13, further comprising a rebuffer heat shrink tube surrounding a stripped area of the optical fiber with a gap between the optical fiber and heat shrink tube filled with potting material.

21. The medical apparatus according to claim 20, wherein the potting material is a dual-cure adhesive inside the rebuffer heat shrink tube only.

22. The medical apparatus according to claim 20, wherein the optical fiber with the rebuffer heat shrink tube is on a guidewire.

23. The medical apparatus according to claim 14, wherein the distal optics comprise at least a spacer, a lens and a reflector.

24. The medical apparatus according to claim 14, wherein the medical apparatus is a disposable catheter.

25. The medical apparatus according to claim 24, wherein the catheter is a multimodal optical coherence tomography catheter.

26. The medical apparatus according to claim 14, wherein the medical device functionally interacts with a display to display images, data or other information.
